(19) United States
(12) Patent Application Publication
(10) Pub. No.: US 2005/0080046 A1
(43) Pub. Date: Apr. 14, 2005

(54) Storage stable eplerenone formulation

(76) Inventors: Pornanong Aramwit, Oak Lawn, IL (US); Hong Qi, Northbrook, IL (US); Leonard J. Ferro, Highland Park, IL (US)

Correspondence Address: PHARMACIA CORPORATION;GLOBAL PATENT DEPARTMENT, POST OFFICE BOX 1027, ST. LOUIS, MO, 63006, US

(21) Appl. No.: 11/001,389

(22) Filed: Dec. 1, 2004

Related U.S. Application Data
(63) Continuation of application No. 10/393,493, filed on Mar. 20, 2003, now abandoned.
(60) Provisional application No. 60/366,064, filed on Mar. 20, 2002.

(51) Int. Cl. A61K 31/724; A61K 31/585

(52) U.S. Cl. 514/58; 514/172

(57) ABSTRACT

There is provided a parenterally deliverable pharmaceutical composition comprising eplerenone and a solvent liquid having the eplerenone in solution therein. Compositions of the invention are storage stable.

This application claims priority to U.S. Provisional application Ser. No. 60/366,064, filed on Mar. 20, 2002.

### FIELD OF THE INVENTION

The present invention relates to pharmaceutical formulations of eplerenone, particularly to ready-to-use, parenterally deliverable eplerenone formulations which are storage stable.

### BACKGROUND OF THE INVENTION

Parenteral drug formulations have become a very important component in the arsenal of available drug delivery options, particularly where a drug is to be administered in a hospital or in-patient setting, and/or where rapid onset of therapeutic effect is desired. Unfortunately, many important drugs exhibit relatively low solubility in water and/or relatively high drug loading and are therefore difficult to formulate as aqueous solutions suitable for parenteral delivery.

Eplerenone (methyl hydrogen 9,11α-epoxy-17α-hydroxy-3-oxopregn-4-ene-7α,21-dicarboxylate, γ-lactone), a steroid, nucleus-based antimineralocorticoid which acts as a competitive inhibitor of aldosterone at aldosterone receptor sites in various tissues, is currently under development by Pharmacia Corp. as an oral tablet formulation for the treatment of hypertension and congestive heart failure. International Patent Publication No. WO 01/41770 discloses that eplerenone has low water solubility making it difficult to formulate as an aqueous solution. That publication also discloses, inter alia, orally deliverable aqueous solutions of eplerenone comprising cylcodextrins. International Patent Publication No. WO 01/41770 further discloses that eplerenone can be formulated for injection.

Eplerenone, first reported in U.S. Pat. No. 4,599,332 to Grob et al., has the structure shown in formula (I):

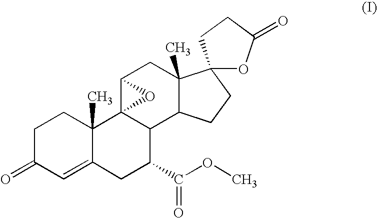

Parenterally deliverable formulations of eplerenone, heretofore not available, would be particularly desirable since many patients with congestive heart failure, hypertension and/or who are recovering from myocardial infarction are unable to take orally deliverable medication on a consistent basis, often for extended time periods. Parenteral eplerenone formulations would also be desirable since such a dosage form could provide a subject with a continuous and level therapeutically effective amount of eplerenone over an extended period of time.

Generally, where parenteral formulation of a drug is desirable, ready-to-use parenteral formulations are particularly advantageous for their ease of administration and reduced risk of contamination or improper dosing (since no mixing is required immediately prior to administration). Moreover, concentrated, and in particular highly concentrated, parenteral dosage forms are also especially desirable for treatment of cardiovascular disorders and related conditions since many such subjects are unable to withstand large volume infusions.

Many parenteral formulations of low water solubility drugs utilize solvents, co-solvents and/or solubilizing agents to dissolve and/or solubilize the drug. Illustrative co-solvents present in parenteral products include polyethylene glycol 400, propylene glycol and glycerin. Additionally, cyclodextrins and derivatives thereof have been used to help solubilize drugs of low water solubility. Sporonox® of Janssen is an illustrative commercially available intravenous product containing itraconazole, an anti-fungal agent, and 40% (w/v) hydroxypropyl-β-cyclodextrin. Sporanox® is indicated for treatment of certain fungal infections. However, since the concentration of hydroxypropyl-β-cyclodextrin is relatively high, Sporanox® is generally not recommended for subjects with renal impairment and is to be administered over a period of not more than 14 days. See e.g. Physicians Desk Reference, Edition 54 (2000), page 1460.

While it is known that eplerenone has low solubility in water, we have now also surprisingly discovered that eplerenone has very low solubility in many organic solvents and/or co-solvents which are commonly used to solubilize drugs in parenteral formulations, even when such solubilizers are in pure form (i.e. not mixed with water). For example, at room temperature, eplerenone has a solubility of less than 0.3 mg/ml, of 3.3-5.7 mg/ml, and of less than 1.6 mg/ml, in pure propylene glycol, polyethylene glycol 400, and glycerin, respectively. Preparation of a pharmaceutically acceptable parenteral eplerenone formulation is therefore made difficult by the fact that (a) the solubility of eplerenone in many otherwise parenterally acceptable solubilizers, even in such solubilizers in pure form, is very low, (b) eplerenone dose requirements are relatively high (e.g. about 50 mg/day or more), and (c) the concentration and/or total amount of many parenterally acceptable solubilizers which can be safely parenterally delivered to a subject is relatively low.

It has also now been surprisingly discovered that preparation of a suitable parenteral eplerenone formulation is further complicated by the fact that stability of eplerenone in aqueous solution is pH-dependent with maximum stability at low pH. Unfortunately, low pH parenteral formulations, particularly where such a formulation is to be administered by intravenous infusion, can be painful to and unsafe for a subject receiving the infusion. Generally, intravenous infusions should not be given at a pH below about 3.5-4.0 so as to minimize local irritation and hemolysis. Furthermore, intravenous administration of acidic solutions having a pH less than about 3.5-4 can cause chemical phlebitis and damage the tunica intima thereby increasing a subject's risk of vein sclerosis, infiltration and thrombosis. Importantly, additional factors such as osmolarity of the formulation and infusion rate also contribute to the presence or absence of such side effects. Nonetheless, this tradeoff between eplerenone stability on the one hand and patient comfort, safety and/or pharmaceutical acceptability on the other has presented practical problems in preparation of a suitable parenteral eplerenone formulation.

If a storage stable, highly concentrated, ready-to-use, parenterally deliverable formulation of eplerenone which could be administered to a subject without unacceptable pain, irritation and/or discomfort could be developed, a significant advance in the treatment of cardiovascular and related diseases and/or disorders would result.

### SUMMARY OF THE INVENTION

The present invention provides a parenterally deliverable pharmaceutical composition comprising a solvent liquid and eplerenone, wherein at least a substantial portion of the eplerenone is in dissolved and/or solubilized form in the solvent liquid and the composition has a pH of about 3.5 to about 6.0. Preferably, the eplerenone is present in an amount of about 0.5 mg/ml to about 100 mg/ml.

In another embodiment, the present invention provides a parenterally deliverable pharmaceutical composition comprising a solvent liquid and eplerenone, wherein at least a substantial portion of the eplerenone is present in the solvent liquid in dissolved and/or solubilized form, the composition has a pH of about 3.5 to about 6.0, and upon storage in a closed container maintained at 25° C. for a period of at least 30 days, at least about 90%, by weight, of eplerenone originally present in the composition is still present therein.

In another embodiment, the present invention provides a parenterally deliverable pharmaceutical composition comprising eplerenone in an amount of about 2.5 mg/ml to about 20 mg/ml, hydroxypropyl-β-cyclodextrin in an amount of about 10 mg/ml to about 110 mg/ml, at least one pharmaceutically acceptable buffering agent, at least one pharmaceutically acceptable isotonic agent, and water. In this embodiment, at least a substantial portion the eplerenone is present in dissolved and/or solubilized form and the composition has a pH of about 3.5 to about 6.0.

In yet another embodiment, the invention provides a parenterally deliverable pharmaceutical composition comprising a solvent liquid and eplerenone, wherein at least a substantial portion of the eplerenone is in dissolved and/or solubilized form in the solvent liquid and wherein the composition comprises means to chemically stabilize the eplerenone. The term “means to chemically stabilize the eplerenone” herein refers to any pharmaceutically acceptable means for preventing, inhibiting, slowing or reducing chemical degradation or reactivity of eplerenone in a composition of the invention.

Compositions of the invention overcome the above-described problems in a surprisingly effective manner. For example, compositions of the invention (a) can be presented as a ready-to-use parenteral formulation requiring no mixing or formulating immediately prior to administration, (b) exhibit excellent eplerenone stability during storage, (c) present a sufficiently high eplerenone concentration so as to allow for relatively small volume administration, and (d) utilize surprisingly low amounts of solubilizing agent and/or other solubilizing excipients so as to be suitable for relatively long-term administration, even over a wide therapeutic dose range.

The term “pharmaceutically acceptable” in relation to an excipient herein means having no persistent detrimental effect on the health of the subject being treated. The pharmaceutical acceptability of an excipient depends on, inter alia, the particular excipient in question, its concentration in the administered composition, and on the route of administration. For example, use of β-cyclodextrin as an excipient in intravenous composition is limited by hemolytic and nephrotoxic effects, but is generally non-toxic when administered orally.

The term “practical limit of solubility” in relation to a drug means the highest concentration at which the drug can be formulated in solution without risk of precipitation or crystallization of the drug during the normal range of manufacturing, packaging, storage, handling and use conditions. Typically the practical limit of solubility is considerably lower than the true solubility limit in a given aqueous medium, for example about 70% of the true solubility limit. Thus, illustratively, for a drug having a true solubility limit in a given aqueous medium of 0.4 mg/ml, the practical limit of solubility is likely to be about 0.28 mg/ml.

Except where the context demands otherwise, use of the singular herein will be understood to embrace the plural. For example, by indicating above that a composition of the invention comprises “a pharmaceutically acceptable cyclodextrin compound”, it will be understood that the composition can contain one or more such cyclodextrin compounds.

The invention also provides a method of preparing a medicament for treating or preventing conditions and/or disorders for which an aldosterone receptor blocker is indicated, using a composition as described herein.

Also embraced by the present invention is a method of treating or preventing conditions and/or disorders for which an aldosterone receptor blocker is indicated, the method comprising administration to the subject of a composition as described herein in a therapeutically and/or prophylactically effective dose. Such administration can be oral, parenteral or topical, but is preferably parenteral and more preferably by intravenous injection or infusion.

### DETAILED DESCRIPTION OF THE INVENTION

Eplerenone

Eplerenone present in a composition of the invention can be prepared according to any suitable method, for example, the methods set forth in U.S. Pat. No. 4,559,332 to Grob et al. and in International Patent Publication No. WO 98/25948 to Ng et al., (particularly scheme 1 set forth therein), both of which disclosures are hereby incorporated herein by reference in their entirety. In one embodiment, eplerenone is present in a composition of the invention at a concentration above the practical limit of solubility of the drug. Preferably, eplerenone is present in a composition of the invention in a total amount of about 0.5 to about 100 mg/ml, more preferably about 1 to about 75 mg/ml, still more preferably about 4 to about 60 mg/ml, and even more preferably about 2.5 mg/ml to about 20 mg/ml. Where the composition is intended for dilution prior to administration or for small volume parenteral administration (e.g. intramuscular or subcutaneous injection), the concentration of eplerenone can be significantly higher, for example about 100 to about 400 mg/ml.

Preferably, at least a substantial portion, more preferably at least about 70%, yet more preferably at least about 80%, even more preferably at least about 90%, still more preferably at least about 95%, and even more preferably substantially all of the eplerenone present in a composition of the invention is in dissolved and/or solubilized form in the solvent liquid.

Solvent Liquid

Compositions of the invention comprise a solvent liquid. The solvent liquid can comprise water, one or more organic or inorganic solvents, one or more co-solvents, one or more solubilizing agents, and/or any other desired pharmaceutically acceptable excipient. Preferably, the solvent liquid is selected so as to maintain eplerenone in solution at the desired eplerenone concentration.

Non-limiting illustrative examples of solubilizing agents, co-solvents, and/or solvents that can be present in the solvent liquid include benzalkonium chloride, castor oil, glyceryl monostearate, lecithin, poloxymer, poloxyethylene fatty acid esters, poloxyethylene stearates, sorbitan esters, stearic acid, cyclodextrins and derivatives thereof, polyethylene glycols, glycerin, propylene glycol, ethanol, dimethylacetamide etc.

Cyclodextrins and derivatives thereof (collectively referred to herein as “cyclodextrin compounds”) are a particularly preferred class of solubilizing agent to be present in the solvent liquid. Exemplary cyclodextrin compounds include α-cyclodextrin, β-cyclodextrin, γ-cyclodextrin, alkylcyclodextrins (e.g., methyl-β-cyclodextrin, dimethyl-β-cyclodextrin, diethyl-β-cyclodextrin), hydroxyalkylcyclodextrins (e.g., hydroxyethyl-β-cyclodextrin, hydroxypropyl-β-cyclodextrin), carboxyalkylcyclodextrins (e.g., carboxymethyl-β-cyclodextrin) and sulfoalkylether cyclodextrins (e.g., sulfobutylether-β-cyclodextrin). More preferred are hydroxyalkyl-β-cyclodextrins and sulfoalkylether-β-cyclodextrins; still more preferred are hydroxypropyl-β-cyclodextrin and sulfobutylether-β-cyclodextrin, with hydroxypropyl-β-cyclodextrin being presently the most preferred.

If desired, complexation of eplerenone by a cyclodextrin compound can be increased by addition of a water-soluble polymer such as carboxymethylcellulose or a salt thereof, hydroxypropylmethylcellulose, or polyvinylpyrrolidone, as described by Loftsson (1998), Pharmazie 53, 733-740.

Particularly preferred cyclodextrin compounds have an average substitution degree (SD) of about 0.1 to about 10, preferably about 1 to about 6 and more preferably about 2.5 to about 5.5. Substituted cyclodextrin compounds can be prepared according to any suitable procedure, for example procedures set forth in U.S. Pat. Nos. 3,459,731 and 4,535,152 and in International Patent Publication No. WO 90/12035, each of which is hereby incorporated herein by reference in its entirety.

If desired, the at least one pharmaceutically acceptable solubilizing agent is present in a total amount of about 0.1 to about 400 mg/ml , preferably about 1 to about 200 mg/ml, and more preferably about 10 to about 150 mg/ml, by weight of the composition. Where the solubilizing agent is a cyclodextrin compound, it is preferably present in an amount effective to enhance solubility of eplerenone, for example in a total amount of about 1 to about 150 mg/ml, more preferably about 5 to about 120 mg/ml, and even more preferably about 10 to about 110 mg/ml. Where the composition is intended for dilution prior to administration, the concentration of cyclodextrin compound can be significantly higher, for example about 100 to about 500 mg/ml. In practice and in view of the high cost of cyclodextrin compounds, the amount of the cyclodextrin compound present in a composition of the invention is preferably only slightly greater, for example no more than about 50% greater and preferably no more than about 20% greater, than the minimum amount required to maintain eplerenone in solution at the desired eplerenone concentration.

In another embodiment, eplerenone and the solubilizing agent(s) are present in a composition of the invention in a weight ratio of about 1:100 to about 1:1, preferably about 1:75 to about 1:1, more preferably about 1:50 to about 1:1, and still more preferably about 1:30 to about 1:1.

Stability, Solubility and pH

The pH at which a drug can be formulated for injection is generally unpredictable for any given drug. For example, levofloxacin for injection USP is prepared to a pH as low as 3.8 while epoprostenol sodium for injection is prepared to a pH as high as 10.8. Furthermore, many individual drugs can themselves be formulated and administered for parenteral delivery over a broad range of pH levels. For example, atropine sulfate for injection USP is said to be administered over a pH range of 3.0-6.5; metoclopramide for injection USP and morphine sulfate for injection USP are said to be administered over a pH range of 2.5-6.5; and mezlocillin sodium for injection USP is said to be administered over a pH range of 4.5 to 8.0; See e.g. Physicians Desk Reference, Edition 54 (2000). However, injectable formulations maintained at isotonic pH of about 3.5 to about 8.5 and preferably 4.0 to about 8.0, are generally considered to be most comfortable, safe and/or tolerable for a subject receiving the formulation, particularly where such a formulation is to be intravenously infused over a substantial period of time.

Surprisingly, we have now discovered that one way to prepare an aqueous eplerenone composition which has acceptable storage stability and which provides suitable safety and comfort upon intravenous administration is to control pH of the composition within a heretofore unpredictable and extraordinarily narrow pH range.

Another way to prepare an aqueous eplerenone composition which has acceptable storage stability and which provides suitable safety and comfort upon intravenous administration is by providing the composition with a solubilizing agent such as a cyclodextrin compound as described more fully herein below.

Yet another way to chemically stabilize eplerenone in a composition of the invention is by maintaining the composition under refrigerated conditions, for example at a temperature of about 30° C. or less, preferably about 25° C. or less, more preferably about 10° C. or less, and still more preferably about 5° C. or less. Without being held to a particular theory, it is believed that when a composition of the invention is formulated above the pH range provided herein (i.e. above about 6.0), substantial eplerenone degradation occurs upon storage of the formulation. Without being bound by theory, it is also believed that one or more of the following degradation compounds, among others, are formed upon storage of such a composition having a pH greater than about 6:
(7α,17α)-9,17-dihydroxy-3-oxopregn-4-ene-7,21-dicarboxylic acid di-γ-lactone (II) 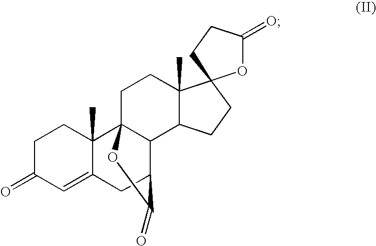
methyl hydrogen (4α,5α,7α,11α,17α)-4,5:9,11-diepoxy-17-hydroxy-3-oxopregnane-7,21-dicarboxylate γ-lactone (III) 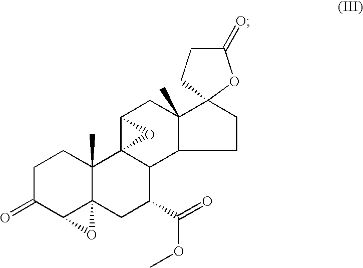
methyl hydrogen (7α,11α,12α,17α)-11,12-epoxy-17-hydroxy-3-oxopregna-4-ene-7,21-dicarboxylate γ-lactone (IV) 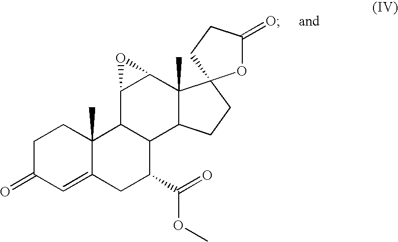
7-methyl hydrogen (7α,11α,17α)-9,11-epoxy-17-hydroxy-3-oxopregn-4-ene-7,21-dicarboxylate (V)

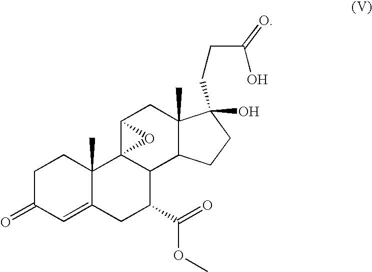

Therefore, in one embodiment, a composition of the invention is prepared to a pH of about 3.5 to about 6.0, preferably about 4.0 to about 5.5, more preferably about 4.1 to about 5.4, more preferably about 4.2 to about 5.3, even more preferably about 4.3 to about 5.2, yet more preferably about 4.4 to about 5.1, and still more preferably about 4.5 to about 5.0.

In another embodiment, a composition of the invention, upon storage in a closed container maintained at 5° C. for a period of at least 30 days, more preferably at least 60 days, yet more preferably at least 90 days, and still more preferably at least 180 days, still contains at least about 90%, preferably at least about 92.5%, more preferably at least about 96%, still more preferably at least about 98%, and even more preferably at least about 99%, by weight, of the eplerenone originally present in the composition. Preferably, upon storage in a closed container maintained at 5° C. for a period of at least 360 days, the composition still contains at least about 90%, preferably at least about 92.5%, and more preferably at least about 96% of the eplerenone originally present in the composition.

In a related embodiment, a composition of the invention, upon storage in a closed container maintained at 25° C. for a period of at least 30 days, more preferably at least 60 days, yet more preferably at least 90 days, and still more preferably at least 180 days, still contains at least about 90%, preferably at least about 92.5%, more preferably at least about 94%, still more preferably at least about 96%, yet more preferably at least about 98%, by weight, of the eplerenone originally present in the composition. Preferably, upon storage in a closed container maintained at 25° C. for a period of at least 360 days, the composition still contains at least about 90%, preferably at least about 92.5%, and more preferably at least about 96% of the eplerenone originally present in the composition.

Improved Patient Convenience and Compliance

It is believed, without being bound by theory, that where a cyclodextrin compound is present in a composition of the invention, enhanced solubility of eplerenone is due, at least in part, to association of at least a portion of the eplerenone with the cyclodextrin compound. It is further believed that at least one mechanism by which the eplerenone associates with the cyclodextrin compound to enhance solubility of eplerenone in an aqueous medium is through formation of an inclusion complex. Such complexes or conjugates are known in the art to form with a variety of drugs, and a number of advantages have been postulated for use of cyclodextrin-drug complexes in pharmacy. See for example review articles by Bekers et al. (1991) in Drug Development and Industrial Pharmacy, 17, 1503-1549; Szejtli (1994) in Medical Research Reviews, 14, 353-386; and Zhang & Rees (1999) in Expert Opinion on Therapeutic Patents, 9, 1697-1717.

Formulations of various drugs with various cyclodextrins have been proposed in the patent literature, including the patents and publications referenced below.

U.S. Pat. No. 5,670,530 to Chen & Shishido discloses compositions comprising a rhodacyanine anti-cancer agent and a cyclodextrin.

U.S. Pat. No. 5,756,546 to Pirotte et al. discloses compositions comprising nimesulide and a cyclodextrin.

U.S. Pat. No. 5,807,895 to Stratton et al. discloses compositions comprising a prostaglandin and a cyclodextrin.

U.S. Pat. No. 5,824,668 to Rubinfeld et al. discloses compositions comprising a 5β steroid drug and a cyclodextrin.

International Patent Publication No. WO 96/32135 discloses compositions comprising propofol and a cyclodextrin.

International Patent Publication No. WO 96/38175 discloses compositions comprising an antiulcerative benzimidazole compound and a branched cyclodextrin-carboxylic acid.

International Patent Publication No. WO 97/39770 discloses compositions comprising a thrombin inhibitor and a cyclodextrin.

International Patent Publication No. WO 98/37884 discloses compositions comprising a 3,4-diarylchroman compound and a cyclodextrin.

International Patent Publication No. WO 98/55148 discloses compositions comprising a sparingly water-soluble drug, a cyclodextrin, a water-soluble acid and a water-soluble organic polymer.

International Patent Publication No. WO 98/58677 discloses compositions comprising voriconazole and a cyclodextrin.

International Patent Publication No. WO 99/24073 discloses compositions comprising a taxoid such as paclitaxel or docetaxel and a cyclodextrin.

International Patent Publication No. WO 99/27932 discloses compositions comprising an antifungal compound of defined formula and a cyclodextrin.

However, the degree of enhancement of solubility achievable through complexation with cyclodextrins of a particular drug or class of drugs is not generally predictable. Cyclodextrin compounds are expensive excipients and in many cases the degree of enhancement of solubility, or other benefit obtained, has not economically justified the increased cost of a formulation arising from addition of a cyclodextrin. The present invention is based in part on the discovery that addition of a modest amount of a cyclodextrin compound increases the solubility of eplerenone to a surprising degree. This enhancement in solubility, among other benefits, makes it possible for the first time to deliver intravenously a therapeutically and/or prophylactically effective dose of eplerenone in a volume small enough to be clinically acceptable and convenient, even for subjects intolerant of large volume intravenous infusion because of hypertension, cardiac, renal and/or other problems. For example, a 50 mg dose of eplerenone can, through use of a composition of the present invention, be delivered intravenously in a volume of 200 ml or less, for example about 20 ml.

Moreover, as indicated hereinabove, currently marketed intravenous dosage forms containing high concentrations (e.g. 40% w/v) of hydroxyproyl-β-cyclodextrin are contra-indicated for subjects with renal insufficiency and are generally not suitable for chronic use. That such extensive enhancement in solubility of eplerenone can be achieved with a surprisingly low concentration (i.e. less than about 30% w/v) of cyclodextrin compound is also beneficial from toxicity, side-effect, and patient compliance standpoints, particularly in instances where such a composition is to be chronically administered (i.e. administered for a periods longer than about 14 days), or administered at a relatively high therapeutic dose.

Additional Excipients

A composition of the invention can further comprise any desired pharmaceutically acceptable excipient. Non-limiting illustrative examples of classes of excipients which may be present include excipients which provide patient comfort (e.g. isotonic agents), further enhance chemical stability (e.g. antioxidants), and/or protect against microorganism growth (e.g. preservatives). Non-limiting examples of suitable anti-microbial agents include phenylmercuric nitrate and thimerosal, benzethonium chloride and benzalkonium chloride, phenol or cresol, and chlorobutabol. Non-limiting illustrative examples of suitable antioxidants include sodium bisuflite, acetone sodium bisulfite, sodium formaldehyde sulfoxylate, and thioourea. Non-limiting illustrative examples of suitable buffers include citrates, acetates and phosphates. Non-limiting illustrative examples of suitable isotonic agents include sodium chloride and dextrose.

Method to Make an Eplerenone Composition

Compositions of the invention can be prepared by any suitable process, including by simple admixture of the ingredients with agitation and/or heat as appropriate. Preferably, where the solvent liquid comprises a cyclodextrin compound, an aqueous solution of the cyclodextrin compound is prepared, and the eplerenone in finely divided solid particulate form is added to that solution with agitation until fully dissolved.

The following is a non-limiting illustrative process for preparing a composition of the invention which comprises a cyclodextrin compound as the solubilizing agent.
A. Water for injection is placed in a vessel;
B. One or more isotonic agents are added to the vessel with agitation to form a mixture;
C. One or more buffers are added to the mixture with agitation;
D. A cyclodextrin compound is added to the mixture with agitation and pH is adjusted to 5.0;
E. Eplerenone is added to the mixture with agitation, and the mixture pH is adjusted to about 5.0;
F. Any additional desired water for injection is added to the mixture;
G. The mixture is sterilized, for example by filtration using a 0.22 mm filter to form a sterile eplerenone composition;
H. The sterile composition is placed into one or more sealed vials and is stored at 5° C. and protected from exposure to UV light until use.
   Utility of Compositions of the Invention

Compositions of the present invention are useful where administration of an aldosterone receptor blocker is indicated. Such compositions are particularly effective in the treatment of cardiovascular diseases such as heart failure; hypertension (especially the management of mild to moderate hypertension); edema associated with liver insufficiency; post-myocardial infarction; cirrhosis of the liver; stroke prevention; and reduction of heart rate for subjects exhibiting an accelerated heart rate. It will be understood that daily dosage amounts provided herein will depend on, inter alia, the particular condition or disorder being treated, and the age, gender, weight, and general health of the subject being treated.

Illustratively, for the treatment of heart failure, a composition of the invention preferably provides a daily dosage of eplerenone in the amount of about 25 mg to about 1000 mg, more preferably about 25 mg to about 200 mg, and still more preferably about 30 to about 100 mg. A daily dose of about 0.33 to about 2.67 mg/kg body weight (based upon an average body weight of about 75 kg), preferably about 0.33 to about 1.00 mg/kg body weight, and most preferably 0.67 mg/kg body weight, is typically appropriate. The daily dose can be administered in about one to about four doses per day, preferably one dose per day.

Illustratively, for the treatment of hypertension, a composition of the invention preferably provides a daily dosage of eplerenone in the amount of about 50 mg to about 1000 mg, more preferably about 50 mg to about 300 mg, and still more preferably about 100 mg. A daily dose of about 0.67 to about 4.00 mg/kg body weight, preferably about 0.67 about 2.00 mg/kg body weight, and most preferably about 1.33 mg/kg body weight, is typically appropriate. The daily dose can be administered in about one to about four doses per day, preferably one dose per day.

Illustratively, for the treatment of edema associated with liver insufficiency, a composition of the invention preferably provides a daily dosage of eplerenone in an amount of about 50 mg to about 1000 mg, more preferably about 100 mg to 400 about mg, and still more preferably about 300 mg. A daily dose of about 0.67 to about 6.67 mg/kg body weight, preferably about 1.33 to about 5.33 mg/kg body weight, and most preferably about 4.00 mg/kg body weight, is typically appropriate. The daily dose can be administered in one to four doses per day, preferably one dose per day.

### EXAMPLES

### Example 1

An eplerenone solution formulation, SF-1, shown in Table 1, was prepared according to the following procedure. Water was placed in a vessel, an isotonic agent (sodium chloride) was added to the water to form a mixture, and the mixture was stirred until the isotonic agent was completely dissolved. A buffering agent (sodium acetate trihydrate) was added to the contents of the vessel and the contents was stirred until the buffering agent was completely dissolved. Hydroxypropyl-β-cyclodextrin (10% wt/vol) was added to the contents of the vessel with stirring, and pH of the contents was adjusted to 5.0 using HCl or NaOH. Eplerenone was added to the contents of the vessel with stirring and the pH was again adjusted to 5.0. A second volume of water was added to the vessel to form an intermediate solution and to bring the total volume of the intermediate solution to a desired final volume. The intermediate solution was filtered using a 0.22 mm filter to form a final solution, and the final solution was transferred into individual vials which were capped with stoppers to form test samples of SF-1.

TABLE 1
Composition (mg) of SF-1

| Component | SF-1 |
|---|---|
| Eplerenone | 5 |
| Hydroxypropyl- | 100 |
| β-cyclodextrin | |
| Sodium acetate | 1.36 |
| trihydrate | |
| Sodium | 9 |
| chloride | |
| Water for | To 1 ml |
| injection | |

### Example 2

Four eplerenone solution formulations, SF-2 to SF-5, were prepared having compositions shown in Table 2. The pH of each formulation was adjusted as indicated.

TABLE 2
Composition of eplerenone solution formulations SF-2 to SF-5

| Component | SF-2 | SF-3 | SF-4 | SF-5 |
|---|---|---|---|---|
| Eplerenone | 0.1 | 0.1 | 0.1 | 0.1 |
| (mg/ml) | | | | |
| Water FI | To 100 | To 100 | To 100 | To 100 |
| PH adjustment | HCl/KCL | 0.01 M acetate | 0.01 M | 0.01 M |
| | | | phosphate | carbonate |
| Final pH | 2 | 5 | 7 | 9 |

Aliquots of each solution formulation were stored at four different temperatures for a period of 12 weeks. Degradation of eplerenone was monitored via high performance liquid chromatography (HPLC) throughout storage. Results are shown in Table 3.

Cells in Table 3 which have the symbol “--” therein indicate that no stability data was collected for that particular time and storage temperature.

TABLE 3
Amount (% weight) of initial eplerenone remaining after
storage at different temperatures and times

| | | | | |
|---|---|---|---|---|
| Time | | | | |
| (days) | 5° C. | 30° C. | 55° C. | 70° C. |

SF-2 (pH 2)

| | | | | |
|---|---|---|---|---|
| 0 | 100 | 100 | 100 | 100 |
| 21 | 99 | 99 | 95 | 71 |
| 42 | 100 | 99 | 88 | 55 |
| 63 | 99 | 98 | 83 | 30 |
| 84 | 100 | 98 | 83 | 27 |

SF-3 (pH 5)

| | | | | |
|---|---|---|---|---|
| 0 | 100 | 100 | 100 | 100 |
| 21 | 99 | 99 | 97 | 88 |
| 42 | 100 | 99 | 97 | 79 |
| 63 | 99 | 98 | 94 | 69 |
| 84 | 99 | 98 | 93 | 55 |

SF-4 (pH 7)

| | | | | |
|---|---|---|---|---|
| 0 | 100 | 100 | 100 | — |
| 6 | — | — | 65 | — |
| 14 | — | 86 | 63 | — |
| 21 | 99 | — | 61 | — |
| 28 | — | 77 | 61 | — |
| 42 | 98 | 71 | — | — |
| 54 | 97 | 64 | — | — |
| 84 | 96 | — | — | — |

SF-5 (pH 9)

| | | | | |
|---|---|---|---|---|
| 0 | 100 | 100 | — | — |
| 1 | — | 44 | — | — |
| 2 | — | 25 | — | — |
| 3 | — | 16 | — | — |
| 6 | 72 | 6 | — | — |
| 14 | 51 | — | — | — |
| 21 | 39 | — | — | — |
| 28 | 30 | — | — | — |

These date show that, during storage at various temperatures and for various time periods eplerenone is more stable in a solution maintained at a pH of 5 than at a pH of 2, 7 or 9.

### Example 3

Two eplerenone solution formulations, SF-6 and SF-7, having compositions shown in Table 4, were prepared according to the general procedure described in Example 1. pH of each solution formulation was adjusted to 5.0 with hydrochloric acid or sodium hydroxide.

TABLE 4
Composition (mg/ml) of SF-6 and SF-7

| Component | SF-6 | SF-7 |
|---|---|---|
| Eplerenone | 5 | 0.5 |
| Hydroxypropyl- | 100 | — |
| β-cyclodextrin | | |
| Sodium acetate | 1.36 | 1.36 |
| trihydrate | | |
| Sodium | — | 9 |
| chloride | | |
| Dextrose | 50 | — |
| Water for | To 1 ml | To 1 ml |
| injection | | |

Individual aliquots of eplerenone solution formulations SF-1 of Example 1 (pH 5), SF-6 and SF-7 were maintained at different temperatures for up to 6 months. As shown in Table 5, amount of initial eplerenone remaining after different storage times was determined by HPLC.

TABLE 5
Amount (% weight) of initial eplerenone remaining after storage
of SF-1, SF-6, and SF-7 at different temperatures and times

SF-1

| | | | |
|---|---|---|---|
| Temperature (° C.) | 5 | 25 | 50 |
| Time (months) | 6 | 3 | 2 |
| Eplerenone | 101.1 | 99.9 | 98.7 |

SF-6

| | | | |
|---|---|---|---|
| Temperature (° C.) | 5 | 25 | 50 |
| Time (months) | 6 | 3 | 2 |
| Eplerenone | 99.2 | 97.6 | 97.2 |

SF-7

| | | | |
|---|---|---|---|
| Temperature (° C.) | 5 | 25 | 50 |
| Time (months) | 6 | 3 | 2 |
| Eplerenone | 68.01 | 63.6 | 67.4 |

These data show that, at pH 5, eplerenone solution formulations comprising a cyclodextrin compound (SF-1 and SF-6) exhibit improved eplerenone stability during storage at various temperatures compared to a comparative eplerenone formulation comprising no cyclodextrin compound (SF-7).

### Example 4

Individual aliquots of SF-1 (pH 5) of Example 1 were stored at 5° C., 25° C., or 50° C. for 6 months. As shown in Table 6. amount of initial eplerenone remaining was determined at different times by HPLC.

TABLE 6
Amount (% weight) of initial eplerenone remaining in
SF-1 after storage at different temperatures and times

| Time | | | |
|---|---|---|---|
| (Months) | 5° C. | 25° C. | 50° C. |
| 1 | 98.4 | 99 | 99.5 |
| 2 | 98.2 | 98.5 | 99.6 |
| 3 | 102.8 | 98.2 | 99.8 |
| 6 | 99.7 | 97.1 | 97.4 |

These data show that eplerenone solution formulation SF-1 exhibits good eplerenone stability during storage at different temperatures over a period of at least 6 months.

### Example 5

A 40 liter batch of eplerenone solution formulation SF-1 of Example 1 was prepared substantially as is described in that example. The solution formulation had an initial pH of about 5.5. Aliquots (10.8 ml) of SF-1 were placed into each of several 10 ml Schott vials and closed with a stopper. Individual vials were stored at 5° C. (ambient relative humidity) or 25° C. (60% relative humidity) for up to 12 months. As shown in Table 7, amount of initial eplerenone remaining after different storage times was determined by HPLC.

TABLE 7
Amount (% weight) of initial eplerenone remaining in each
vial after storage for up to 12 months

| Time | | |
|---|---|---|
| (Months) | 5° C. | 25° C. |
| 0 | 98.1 | 98.1 |
| 1 | 96.7 | 98.2 |
| 3 | 98.4 | 97.9 |
| 6 | 98.6 | 98.5 |
| 12 | 97.6 | 97.9 |

As is shown in Table 7, eplerenone solution formulation SF-1 exhibited excellent stability during storage at both 5° C. and 25° C. for up to 12 months.

1. A parenterally deliverable pharmaceutical composition comprising a solvent liquid and eplerenone, wherein at least a substantial portion of the eplerenone is present in the solvent liquid in dissolved and/or solubilized form and the composition has a pH of about 3.5 to about 6.0.

2. The composition of claim 1 wherein the eplerenone is present in a therapeutically and/or prophylactically effective amount.

3. The composition of claim 1 wherein the eplerenone is present in an amount of about 0.5 mg/ml to about 100 mg/ml.

4. The composition of claim 1 wherein the eplerenone is present in an amount of about 1 mg/ml to about 75 mg/ml.

5. The composition of claim 1 wherein the eplerenone is present in an amount of about 4 mg/ml to about 60 mg/ml.

6. The composition of claim 1 having a pH of about 4.0 to about 5.5.

7. The composition of claim 1 having a pH of about 4.2 to about 5.3.

8. The composition of claim 1 having a pH of about 4.3 to about 5.2.

9. The composition of claim 1 wherein the solvent liquid comprises at least one pharmaceutically acceptable solubilizing agent.

10. The composition of claim 9 wherein the at least one solubilizing agent is present in a total amount of about 0.1 to about 400 mg/ml.

11. The composition of claim 9 wherein the at least one solubilizing agent is present in a total amount of about 1 to about 200 mg/ml.

12. The composition of claim 9 wherein the at least one solubilizing agent is present in a total amount of about 10 to about 150 mg/ml.

13. The composition of claim 9 wherein the at least one solubilizing agent is a cyclodextrin compound.

14. The composition of claim 13 wherein the cyclodextrin compound is selected from the group consisting of α-cyclodextrins, β-cyclodextrins, γ-cyclodextrins, alkylcyclodextrins, hydroxyalkylcyclodextrins, carboxyalkylcyclodextrins and sulfoalkylether cyclodextrins.

15. The composition of claim 13 wherein the cyclodextrin compound is selected from the group consisting of methyl-β-cyclodextrin, dimethyl-β-cyclodextrin, diethyl-β-cyclodextrin, hydroxyethyl-β-cyclodextrin, hydroxypropyl-β-cyclodextrin, carboxymethyl-β-cyclodextrin, and sulfobutylether-β-cyclodextrin.

16. The composition of claim 13 wherein the cyclodextrin compound is hydroxypropyl-β-cyclodextrin.

17. The composition of claim 13 wherein the cyclodextrin compound is present in a total amount of about 1 mg/ml to about 150 mg/ml.

18. The composition of claim 13 wherein the cyclodextrin compound is present in a total amount of about 5 mg/ml to about 120 mg/ml.

19. The composition of claim 13 wherein the cyclodextrin compound is present in a total amount of about 10 mg/ml to about 110 mg/ml.

20. The composition of claim 1 wherein, upon storage of the composition in a closed container maintained at 25° C. for a period of at least 30 days, said eplerenone constitutes at least about 90%, by weight, of eplerenone originally present in the composition.

21. The composition of claim 1 wherein, upon storage of the composition in a closed container maintained at 25° C. for a period of at least 60 days, said eplerenone constitutes at least about 98%, by weight, of eplerenone originally present in the composition.

22. The composition of claim 1 wherein, upon storage of the composition in a closed container maintained at 25° C. for a period of at least 90 days, said eplerenone constitutes at least about 96%, by weight, of eplerenone originally present in the composition.

23. The composition of claim 1 wherein, upon storage of the composition in a closed container maintained at 25° C. for a period of at least 180 days, said eplerenone constitutes at least about 96%, by weight, of eplerenone originally present in the composition.

24. The composition of claim 1 wherein, upon storage of the composition in a closed container maintained at 25° C. for a period of at least 360 days, said eplerenone constitutes at least about 96%, by weight, of eplerenone originally present in the composition.

25. A parenterally deliverable pharmaceutical composition comprising a solvent liquid and eplerenone, wherein at least a substantial portion of the eplerenone is present in the solvent liquid in dissolved and/or solubilized form, the composition has a pH of about 4.0 to about 5.5, and upon storage in a closed container maintained at 25° C. for a period of at least 180 days, said eplerenone constitutes at least about 96%, by weight, of eplerenone originally present in the composition.

26. The composition of claim 25 wherein the eplerenone is present, at time of preparation of the composition, in an amount of about 0.5 mg/ml to about 100 mg/ml.

27. The composition of claim 25 wherein the eplerenone is present, at time of preparation of the composition, in an amount of about 4 mg/ml to about 60 mg/ml.

28. The composition of claim 25 having a pH of about 4.2 to about 5.3.

29. The composition of claim 25 having a pH of about 4.3 to about 5.2.

30. The composition of claim 25 wherein the solvent liquid comprises at least one pharmaceutically acceptable solubilizing agent.

31. The composition of claim 30 wherein the at least one solubilizing agent is present in a total amount of about 0.1 to about 400 mg/ml.

32. The composition of claim 30 wherein the at least one solubilizing agent is present in a total amount of about 10 to about 150 mg/ml.

33. The composition of claim 30 wherein the at least one solubilizing agent is a cyclodextrin compound.

34. The composition of claim 33 wherein the cyclodextrin compound is selected from the group consisting of α-cyclodextrins, β-cyclodextrins, γ-cyclodextrins, alkylcyclodextrins, hydroxyalkylcyclodextrins, carboxyalkylcyclodextrins and sulfoalkylethercyclodextrins.

35. The composition of claim 33 wherein the cyclodextrin compound is selected from the group consisting of methyl-β-cyclodextrin, dimethyl-β-cyclodextrin, diethyl-β-cyclodextrin, hydroxyethyl-β-cyclodextrin, hydroxypropyl-β-cyclodextrin, carboxymethyl-β-cyclodextrin, and sulfobutylether-β-cyclodextrin.

36. The composition of claim 33 wherein the cyclodextrin compound is hydroxypropyl-β-cyclodextrin.

37. The composition of claim 33 wherein the cyclodextrin compound is present in a total amount of about 1 mg/ml to about 150 mg/ml.

38. The composition of claim 33 wherein the cyclodextrin compound is present in a total amount of about 10 mg/ml to about 110 mg/ml.

39. A parenterally deliverable pharmaceutical composition comprising eplerenone in an amount of about 2.5 mg/ml to about 20 mg/ml, hydroxypropyl-β-cyclodextrin in an amount of about 10 mg/ml to about 110 mg/ml, at least one pharmaceutically acceptable buffering agent, at least one pharmaceutically acceptable isotonic agent, and water; wherein at least a substantial portion the eplerenone is present in dissolved and/or solubilized form and the composition has a pH of about 3.5 to about 6.0.

40. A method of treating and/or preventing a condition or disorder where an aldosterone receptor blocker is indicated, comprising parenterally delivering to a subject in need of such treatment and/or prevention a therapeutically effective amount of a composition of claim 1.

41. A method of treating and/or preventing a condition or disorder where an aldosterone receptor blocker is indicated, comprising parenterally delivering to a subject in need of such treatment and/or prevention a therapeutically effective amount of a composition claim 25.

42. A method of treating and/or preventing a condition or disorder where an aldosterone receptor blocker is indicated, comprising parenterally delivering to a subject in need of such treatment and/or prevention a therapeutically effective amount of a composition claim 39.

43. The method of any one of claims 40, 41 or 42 wherein the condition or disorder is selected from the group consisting of heart failure, hypertension, edema associated with liver insufficiency, myocardial infarction, cirrhosis of the liver, and stroke.
